(19) United States
(12) Patent Application Publication
(10) Pub. No.: US 2026/0191636 A1
(43) Pub. Date: Jul. 9, 2026

(54) A COMPOSITE COMPLIANT VASCULAR GRAFT

(71) Applicant: ASCENSE MEDICAL GMBH, Vienna (AT)

(72) Inventors: Jeremy Douglas JARMAN, Vienna (AT); Smurti MIRCHANDANI, Johannesburg (ZA); Michael Matthew MOORE, Miami Beach, FL (US); Johanna Maria TICAR, Vienna (AT)

(21) Appl. No.: 19/133,586

(22) PCT Filed: Mar. 22, 2024
(86) PCT No.: PCT/IB2024/052753

§ 371 Date: May 28, 2025

(30) Foreign Application Priority Data
Mar. 22, 2023 (ZA) 2023/00507

(51) Int. Cl. A61F 2/07; A61F 2/06; D02G 3/32; D02G 3/34; D02G 3/38; D02G 3/44; D03D 15/47; D03D 15/56; D04B 21/12; D04B 21/14; D04B 21/20

(52) U.S. Cl. CPC A61F 2/07; D02G 3/32; D02G 3/34; D02G 3/38; D02G 3/448; D03D 15/47; D03D 15/56; D04B 21/12; D04B 21/14; D04B 21/20; A61F 2002/068; A61F 2002/072; A61F 2210/0076; D10B 2509/06

(57) ABSTRACT

The invention disclosure provides a composite compliant vascular graft for implantation in a host, which graft includes a tubular body having a lumen and a tubular wall surrounding the lumen made of a composite material, which is adapted to permit expansion of the tubular body from a native state to an expanded state and then to return to the native state in response to pressure changes in the lumen, which includes a mechanical component which is adapted to limit the expanded state, and a compliant component which is adapted to bias return of the tubular body to the native state.

This application is a National Phase entry under § 371 of International Application No. PCT/IB2024/052753 and claims the benefit of South African Patent Application No. 2023/00507, filed on Mar. 22, 2023, which is hereby incorporated by reference in its entirety.

### FIELD OF THE DISCLOSURE

This disclosure relates to a composite compliant vascular graft material used in surgical vascular grafts and endovascular grafts for the treatment of compliant vessels.

### BACKGROUND

Existing surgical vascular grafts and endovascular grafts are predominantly designed to treat vascular diseases by providing structures such as generally tubular bodies, tapered or conical bodies, bifurcated bodies, tubular bodies with fenestrations, and/or bodies with side branches. These structures act as artificial vessels, serving to replace or exclude the affected vessels. The specific function of a surgical or endovascular vascular graft in treating vascular disease determines its specific form.

The graft bodies employed in aortic surgical and endovascular grafts are typically constructed from woven or knitted fabrics in a matrix form, utilizing suitable plastic materials like polyethylene terephthalate (PET) or spun fibers of polytetrafluoroethylene (PTFE).

The described grafts are non-compliant, both radially and longitudinally, due to the woven or knitted matrix nature of the body.

A typical surgical graft, which is tubular and/or conical, is crimped or pleated to provide cylindrical stability and flexibility when placed in a curved position. These grafts have proximal and distal ends that are end-to-end anastomosed to bridge a removed segment of or lay within the diseased vessel of the patient. These surgical vascular devices are radially non-compliant.

A typical endovascular graft, commonly used to treat an aneurysm, consists of proximal and distal anchor segments with multiple circumferentially expansive stent elements engaged to the tubular body's wall. These stent elements are spaced at longitudinal intervals, ending at the proximal and distal ends, and are adapted to anchor in healthy aortic tissue proximal (referred to as “proximal landing zone”) and distal (referred to as “distal landing zone”) to the aneurysm. This placement allows the vascular graft to form a seal within the aorta, with the medial portion of the vascular graft bridging the aneurysm and excluding it from the circulating blood flow.

Each stent element is typically a nitinol ring that allows for circumferential expansion and compression. However, the oversized nature of the tubular graft body and each stent element, relative to the aorta's diameter, imparts a significant radial force on the aorta and causes infolding or pleating of the tubular graft body upon implantation.

To achieve an effective seal, an oversizing of the vascular graft relative to the aortic diameter at the proximal and distal landing zones is required, with a typical oversizing factor of 10%-20% chosen for aneurysms.

These vascular grafts are also used in the treatment of aortic dissections, but the physiological differences from an aneurysm pose challenges. A dissection is caused by a tear in the intimal layer of the aorta, leading to blood leakage and the formation of a “false lumen” separate from the “true lumen.” Endovascular treatment aims to occlude the entry tear and prevent blood flow into the wall between vessel wall layers.

However, the inherent rigidity of these grafts reduces the “windkessel function” of the aorta, potentially inducing left ventricular hypertrophy, increasing blood pulse-wave velocity, and leading to arterial hypertension and higher blood pressure.

Furthermore, lack of compliance can result in mechanical complications such as retrograde dissection or distal vascular graft induced new entries (dSINE), necessitating open surgery or secondary vascular graft placement.

The present disclosure at least partially addresses the problem.

### SUMMARY

The disclosure provides a composite compliant vascular graft for implantation in a host, which graft includes a tubular body having a lumen and a tubular wall surrounding the lumen made of a composite material, which is adapted to permit expansion of the tubular body from a native state to an expanded state and then to return to the native state in response to pressure changes in the lumen, which includes a mechanical component which is adapted to limit the expanded state, and a compliant component which is adapted to bias return of the tubular body to the native state.

The mechanical component may be adapted to limit an increase in a radial dimension of the expanded state.

The mechanical component may be adapted to limit a decrease in a longitudinal dimension of the expanded state.

The mechanical component may be adapted to accommodate an increase in the longitudinal dimension of the expanded state.

The mechanical component may comprise a framework of filamentous elements.

The filamentous elements may be yarns which include elastic yarns, inelastic yarns, or a combination of elastic and inelastic yarns (composite yarns).

The yarns may have a linear density of from 10 dtex to 80 dtex.

The elastic yarns may be made of filaments of a thermoplastic polyurethane or polyurethane-polyurea copolymer material, and the inelastic yarns are made of filaments of a polyester or nylon.

The yarns may be adapted (by forming or by construction) to elongate under load to an elongation limit.

The composite yarns may include elastic yarns with inelastic yarns twisted around the elastic yarns.

The inelastic yarns may twist around the elastic yarns in a twist density of from 25 to 1500 twists per meter.

The yarns may be formed with folds, or the yarns may be textured to release constituent filaments as wisps.

The framework may be a matrix of yarns intertwined in a knit structure or a weave structure.

The weave structure may comprise weft yarns and warp yarns which are adapted to adjust the extent of radial expansion or longitudinal dimension of the expanded state.

The weft yarns may be composite yarns.

Alternatively, the weft and the warp elements may be composite yarns.

The twist density of the weft yarns may differ from the twist density of the warp yarns.

The knit structure is a warp knit or a weft knit.

The warp knit may include a plurality of wales and a plurality of connecting elements, each connecting a pair of wales.

Each wale may comprise a pillar of stiches formed by a warp yarn of a first set, and each connecting element is an inlay yarn of a first set.

Alternatively, the knit structure may be created with a tricot stitch pattern in which each wale comprises stitches alternately formed by a warp yarn of a first set and a warp yarn of a second set, and the connecting elements are cross-links generated by each warp yarn as it extends between a pair of wales.

The inlay yarns of the first set may adopt a sinusoidal pattern as they traverse between and link pairs of adjacent wales.

The warp knit may include inlay yarns of a second set.

The inlay yarns of both the first and second sets adopt a sinusoidal pattern as they interlace with the wales from opposing directions, connecting pairs, and wherein the inlay yarns of both the first and second sets extend in opposite directions, aligning the peaks of the yarns in the first set with the troughs of the yarns in the second set.

The pairs of wales that are connected by the inlay yarns may be adjacent one another or separated by one or more interposing wales.

The inlay yarns of the first and second sets may extend in phase, with the peaks of the yarns in the first set aligning with the peaks of the yarns in the second set.

The inlay yarns of the first and second sets may extend out of phase, with the peaks of the yarns in the first set aligning with the troughs of the yarns in the second set.

The inlay yarns of the first and the second sets may be inelastic yarns.

Further alternatively, the warp knit structure may form a symmetrical net, wherein each wale is intertwined alternatively with an adjacent wale on one side and with an adjacent wale on the opposite side to provide intertwined sections, and wherein between these sections, there is a gap where the wales are not intertwined.

The gaps may provide net openings which are hexagonal in shape.

The net openings may be dog bone shaped by one of more of the following: longitudinal compression and heat setting.

The warp knit may have a latitudinal knit density of between 3 and 50 wales per cm.

The warp knit may have a longitudinal knit density of between 6 and 50 courses per cm.

The compliant component may comprise at least a first layer of the tubular wall that is engaged to an underlayer of the mechanical component or that encapsulates the mechanical component.

The compliant component may comprise a tie layer of the tubular wall that is engaged to an underlayer being the first layer or the mechanical component.

The compliant component may comprise at least a second layer of the tubular wall that is engaged to an underlayer being the first layer, the tie layer, or the mechanical component.

The first or the second layer may be made of a polycarbonate thermoplastic polyurethane (polycarbonate TPU), or a thermoplastic polyurethane silicone co-polymer (TPU-Silicones).

The tie layer may be made of a polyester or polyether thermoplastic polyurethane or an aliphatic or aromatic di-isocyanate thermoplastic polyurethane.

The TPU-Silicones may include silicone in a concentration of 5%-50% (wt/wt).

The polycarbonate TPU or TPU-Silicones may have a Shore hardness of from 65A to 95 A

The polyester or polyether thermoplastic polyurethane, the aliphatic di-isocyanate thermoplastic polyurethane, or aromatic di-isocyanate thermoplastic polyurethane may have a Shore hardness of from 42A to 70A.

The first layer or the second layer may include longitudinal zones of varying durometer.

The longitudinal zones may include proximal and distal zones and an intermediate zone between the proximal and distal zones.

The polycarbonate TPU or TPU-Silicones of the proximal and distal zones may have a Shore hardness of from 85A to 95 A and the polycarbonate TPU or TPU-Silicones of the intermediate zone may have a Shore hardness of from 65A to 85 A.

The compliant component may be a preform which is engaged as the first layer, second layer or tie layer to the underlayer.

Alternatively, the compliant component may be applied directly as the first layer, second layer or tie layer to the underlayer to cover or to encapsulate the underlayer.

The compliant component may be a laminar layer or a filamentous layer.

The filamentous layer may include an inner layer, or outer layer, comprising of filaments orientated in multiple directions and an outer layer, or inner layer respectively, comprising of filaments orientated unidirectionally.

The filaments of the outer layer or the inner layer may be orientated in a circumferential or an axial direction of the tubular body.

The composite compliant vascular graft may include a brace which engages an exterior of the tubular body, and which is adapted to limit the radial dimension of the expanded state.

The brace may include at least one yarn which is inelastic, or which is a combination of elastic and inelastic yarns that are wrapped around the tubular body.

The brace may be a tubular frame made of an elastic polymeric material or a super elastic metal alloy such as, for example, nitinol.

The brace may comprise a plurality of rings, each made of a super elastic metal alloy.

### BRIEF DESCRIPTION OF THE DRAWINGS

The disclosure is further described by way of examples with reference to the accompanying drawings in which

FIG. 1 isometrically illustrates a composite compliant vascular graft in accordance with the disclosure,

FIG. 2 is a view in cross section of the composite compliant vascular graft in accordance with a first embodiment of the invention,

FIG. 3 is a view in cross section of the composite compliant vascular graft in accordance with a second embodiment of the invention,

FIG. 4 is a view in cross section of the composite compliant vascular graft in accordance with a third embodiment of the invention,

FIG. 5A is a view in cross section of the composite compliant vascular graft in accordance with a fourth embodiment of the invention,

FIG. 5B is a isometric, longitudinally sectioned, view of the vascular graft of FIG. 5A,

FIG. 6 is a view in cross section of the composite compliant vascular graft in accordance with a fifth embodiment of the invention,

FIG. 7A is a graphical representation of the relationship between pressure within the vascular graft, and the increase in its diameter,

FIG. 7B is a graphical representation of the correlation between stress and strain experienced by the vascular graft,

FIG. 8A schematically illustrates a composite yarn used in some examples of the vascular graft, in a unstretched configuration,

FIG. 8B schematically illustrates a composite yarn used in some examples of the vascular graft, in a stretched configuration,

FIG. 9 illustrates a textured yarn used in some examples of the vascular graft,

FIG. 10 presents an isometric, plan view of a pillar stitch knit pattern of a knitted fabric used in some examples of the vascular graft,

FIGS. 11A and 11B each present an isometric, slanted view of the pillar stitch knit pattern of a knitted fabric of FIG. 10 in a relaxed configuration and a stretched configuration respectively,

FIG. 12 is a lapping diagram representing the knit pattern of FIGS. 10, 11A and 11B,

FIGS. 13, 14 and 15 are lapping diagrams representing various alternative inlay patterns introduced to a tricot knit pattern of a knitted fabric used in some examples of the vascular graft,

FIG. 16 is a lapping diagrams representing an inlay patterns introduced to a tricot knit pattern of a knitted fabric used in some examples of the vascular graft,

FIGS. 17A, 17B and 17c each illustrate an isometric, plan view of the tricot stitch knit pattern of a knitted fabric of FIG. 16,

FIGS. 18A and 18B each present an isometric, plan view of the tricot stitch knit pattern of a knitted fabric of FIGS. 16 and 17, in a relaxed configuration and a stretched configuration respectively,

FIG. 19 presents an isometric, plan view of an auxetic knit pattern of a knitted fabric used in some examples of the vascular graft,

FIG. 20 is a lapping diagram of the auxetic knit pattern of a knitted fabric of FIG. 19,

FIG. 21 is an isometric, plan view of a variation to the auxetic knit pattern of a knitted fabric shown in FIG. 19,

FIGS. 22A and 22B schematically illustrate a weave pattern of a woven fabric used in some examples of the vascular graft, in a relaxed and a stretched configuration respectively,

FIG. 23 schematically illustrates an inlay variation on the pillar stitch knit pattern of a knitted fabric of FIG. 10,

FIGS. 24 and 25 schematically illustrate variations on the woven fabric of FIGS. 22A and 22B,

FIG. 26 isometrically illustrates a composite compliant vascular graft in accordance with a sixth embodiment of the invention,

FIG. 27 isometrically illustrates a composite compliant vascular graft in accordance with a seventh embodiment of the invention,

FIGS. 28A and 28B provide schematic illustrations of the fiber layering constituting the outer sub-layers of the vascular graft depicted in FIG. 27, and

FIGS. 29A through 29E depict isometric illustrations of vascular grafts in accordance with the disclosure, each showcasing a variation in the composition, configuration, or pattern of an outer brace.

### DETAILED DESCRIPTION

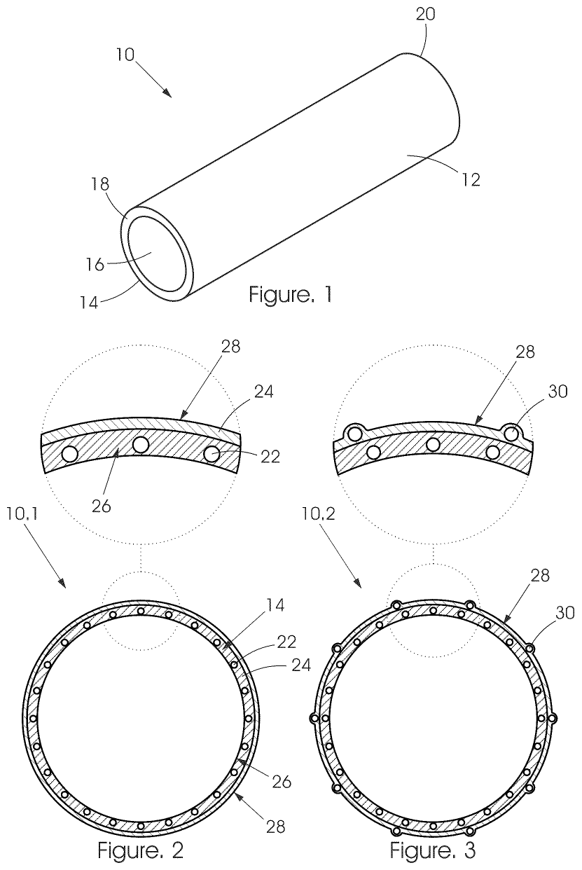

FIG. 1 of the accompanying drawings illustrates a composite compliant vascular graft 10 in accordance with a first embodiment of the invention, for use in surgical grafts and endovascular vascular grafts for the treatment of compliant vessels.

The treatment can be surgical or endovascular aortic or peripheral vascular repair.

The vascular graft 10 has a tubular body 12 having a tubular wall 14, made of a composite material, surrounding, and defining a lumen 16. In the longitudinal direction, the body extends between a proximal end 18 and a distal end 20.

Referring to FIG. 2, the wall 14 of vascular graft body 12 is composite in that it has a mechanical component 22 and a compliant component 24. In this example 10.1, the mechanical component, a fabric frame, is embedded in the compliant component. This example, the compliant component is layered, comprising a first layer 26 and a second layer 28 coating the first layer.

The tubular body is designed to contract and expand harmoniously within or across the vessel, synchronizing with the fluctuation of pressure within the lumen. This pressure varies between a native state, observed at a diastolic minimum, and an expanded state, noted at a systolic maximum. The tubular body's composite composition allows for permissive and compliant movement between these two states, preserving the vessel's windkessel function.

To be functionally relevant, the tubular body is adapted to expand by between 5% to 30% of the diameter of the native state. In a preferred embodiment, this radial compliance is integrated with longitudinal compliance, allowing the tubular body to undergo longitudinal strain of up to 32%. The graph of FIG. 7B illustrates the stress, strain relationship in radial compliance, and the inherent limitation on elasticity as a result of the disclosure.

The compliant component is designed to impart compliance to the tubular body, enabling this expansion and facilitating the biased return from the expanded state to the native state. At the same time, the mechanical component is configured to restrict the expanded state while, preferably, preventing foreshortening of the tubular body in the longitudinal direction during radial expansion. The graph of FIG. 7A illustrates this relationship between pressure and diametric expansion of the graft before reaching a restricted limit. This limitation can be important in preventing the graft from exceeding hypertensive limits, which could surpass the elastomeric range of the compliant component.

The mechanical component is a framework of filamentous elements, more precisely, a fabric which can be a knitted or woven fabric comprising elastic, non-elastic, or composite yarns.

The first layer 26 of the compliant component is an elastic polymeric material. The second layer 28 also comprises an elastic polymeric material. The composition of the elastic polymeric material of the first and second layers can differ, with the first layer providing properties that dictate the device compliance while the second layer primarily is formulated to retain stent forms, provide erosion resistance and/or other beneficial properties.

To keep the tubular form of the tubular body, the graft 10.2 can include stents 30 such as, for example, stents of a waveform shape made of a super-elastic material such as nitinol. This example is shown in FIG. 3, where a waveform stent is shown encapsulated within the second layer 28.

The polymeric material of the first or second layer can be a polyurethane, a class of materials chosen for their elastomeric liquid-tight properties, enabling radial compliance whilst restricting permeability of the tubular wall to blood that would flow across the wall due to any porosity of the underlayers.

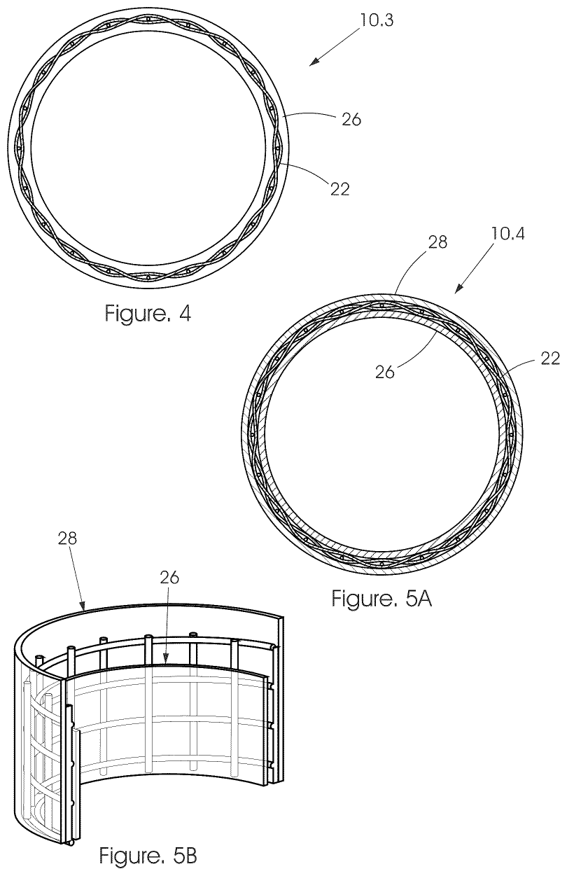

In another example of the vascular graft 10.3, shown in FIG. 4, the wall 14 only has a single layer 26 of the compliant component 24, with the mechanical component 22 embedded within this layer.

In the example depicted in FIGS. 5A and 5B, the wall of the vascular graft 10.4 comprises a compliant first internal layer 26 and a compliant second external layer 28. The mechanical component 22 is represented by a discrete layer sandwiched between the internal and external layers. The layers are laminated to one another, i.e. bonded together to form a single composite structure, by any suitable process such as, for example, adhesive bonding, heat, pressure, or a combination of these.

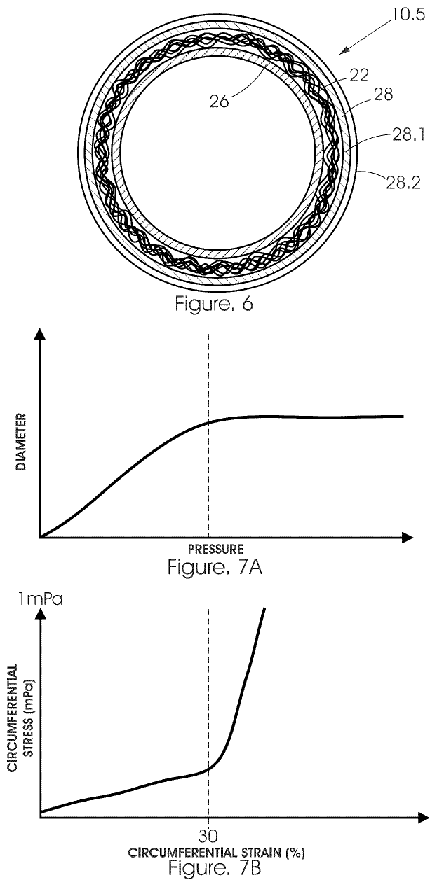

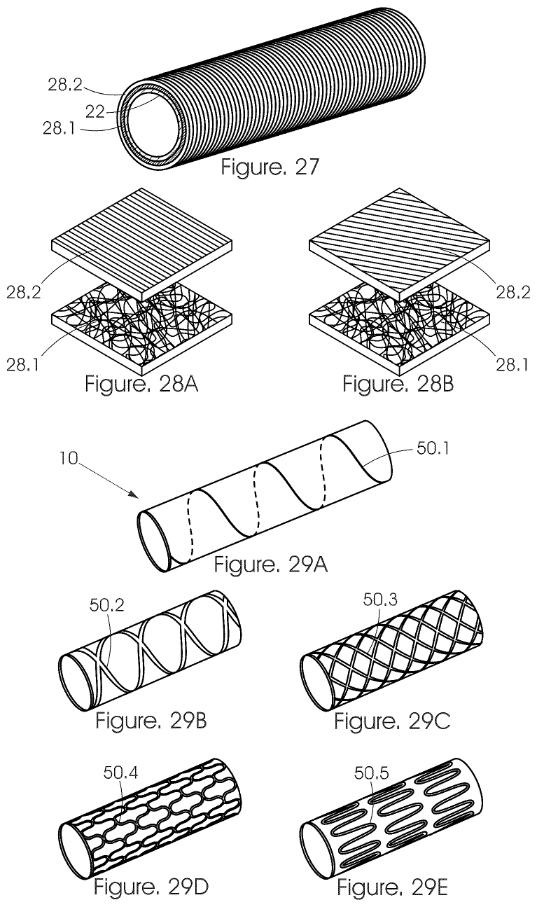

The iterative process persists, as demonstrated in FIG. 6, showcasing yet another example. In this instance, the vascular graft 10.5 maintains a mechanical component 22 positioned between an internal and an external layer (26, 28). Notably, in this embodiment, the compliant component's external layers consist of two sub-layers (28.1 and 28.2), distinguishing it from the previously described example. The Layers can differ in their relative adhesive properties, with layer 28.1 being a layer with relative adhesive properties, adhering to and around the mechanical component, known as a tie layer, onto which layer 28.2 can be laminated. Alternatively, the layers can differ in their configuration, with both layers being filamentous compliant layers (see FIGS. 28, 29A and 29B) but with layer 28.1 having the filaments laid down randomly, with the filaments of layer 28.2 laid down in alignment as will be more fully described below.

Several key attributes, either independently or in conjunction, configure the mechanical aspect to enable, at least, radial expansion while simultaneously imposing a strain-stiffening constraint as the expanded state approaches its operational limit (see FIG. 7). These attributes, referred to as “lock-out” features, encompass fabric type (such as whether knitted or woven), fabric pattern (the arrangement within the knitted or woven fabric), yarn type (whether elastic, inelastic, or composite), and yarn structure (whether composed of straight or formed filaments, or textured). Generally, the mechanical component undergoes conformational changes upon approaching the limiting diameter, which provide resistance to further expansion.

### Yarns

As mentioned, the fabric (the mechanical component) can be knitted or woven. The yarns making up this fabric can be elastic, non-elastic, or composite.

The elastic yarns, in the examples that follow, are made of TPU or polyurethane-polyurea copolymer. The inelastic yarns are made of PET or Nylon 6,6.

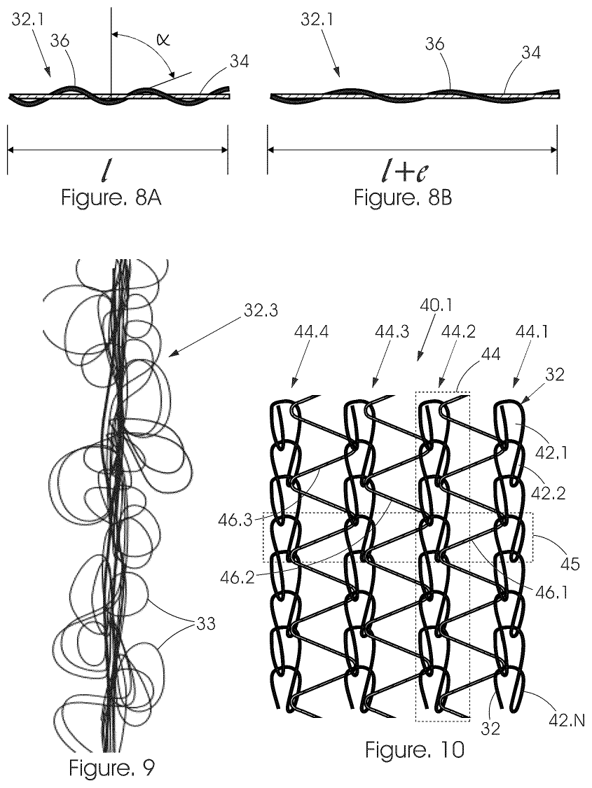

With reference to FIGS. 8A and 8B, a composite yarn 32.1 includes an elastic core yarn 34, wrapped with at least one inelastic cover yarn 36. The composite yarn (herein called a “covered yarn”) can be adapted to achieve a desired elongation or strain before the inelastic cover yarn is engaged, altering the cover angle α, to limit further elongation. Modifying this angle affects the twists per meter, influencing the amount of covering yarn applied over the core and determining the resulting slack in the yarn. A decreased cover angle, leading to more twists per meter, will result in greater elongation before engagement. This lock-out feature is illustrated (FIG. 8B) in the stretched configuration of the yarn.

The composite yarn can be customized to meet specific functional requirements by modifying one or more of the following parameters: the material of the elastic core yarn and the inelastic cover yarn, the number of core and cover yarns in the composite yarn, the linear density of the core and the cover yarn, the twist density and the inherent structure or texture of the yarn.

The twist density is the amount of times that the cover yarn of each composite yarn twists around the core yarn, per meter. In the context of the disclosure, the range is from 25 to 1500 twists per meter.

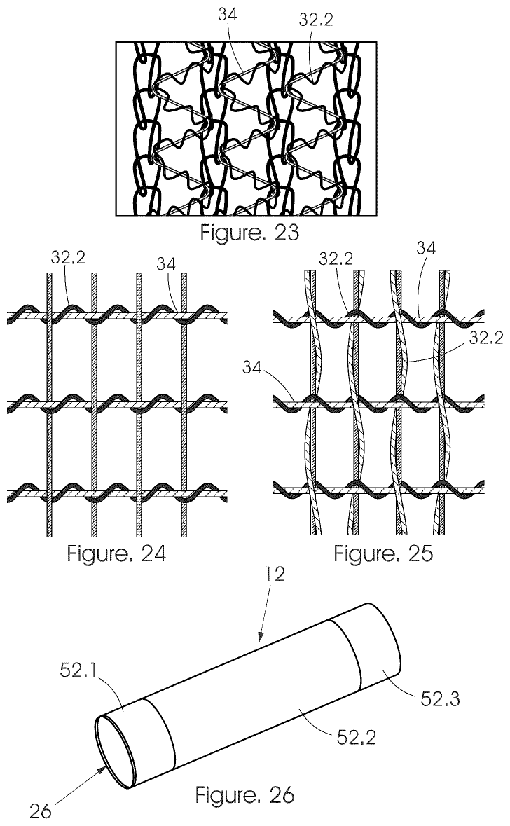

In terms of its intrinsic structure or texture, yarns, preferably inelastic, can undergo processing such as knitting and de-knitting, or heat setting to provide a crimped yarn 32.2 (see FIGS. 23 to 25), or air texturizing to provide a textured yarn 32.3. FIG. 9 illustrates the textured outcome of air texturizing on the yarn 32.3, revealing numerous “released” filaments 33.

Crimped yarns 32.2 can be introduced into a knit fabric, such as that illustrated in FIG. 23, in which the crimped yarn is the cover yarn which covers the core yarn 34 to provide an alternative to the standard composite yarn (32.1) described above. The crimping of the crimped yarn capacitates this composite yarn with additional inherent capacity to elongate before locking out.

FIG. 23 illustrates the introduction of the crimped yarn 32.2, as a component of a composite inlay yarn, into a knit. FIGS. 24 and 25 illustrate the introduction of the crimped yarn 32.2, as a component of a composite inlay yarn, into a woven fabric in the weft direction (FIG. 24) and in both warp and weft directions (FIG. 25).

### Knits

While the fabric can be manufactured using both warp knitting and weft knitting techniques, the disclosure is not limited to these methods. Nevertheless, a preference is expressed for warp knitted fabric over weft knitted fabric, as warp patterns offer greater customization for finely tuning the mechanical component's functionality.

In the subsequent examples of warp knit fabrics, a common feature is the interconnectedness of the wales through connecting elements, enabling radial expansion of the fabric. However, what varies among them is the composition, configuration, or source of these connecting elements.

Pillar Stitch: In a first example of a knitted fabric 40.1, illustrated in FIG. 10, the knit pattern is a pillar stitch knit pattern. In this pattern, each warp element is a yarn 32 stitched into a column of loops or stitches (the loops are designated 42.1, 42.2, . . . 42.N), called a wale, that forms in the longitudinal direction of the tubular body 12. The wales are respectively designated 44.1, 44.2, 44.3 and 44.4. A series of courses, each comprised of a row loops (one course is highlighted in dotted outline, designated 45), extend in the radial direction. The wales are connected with a plurality of connecting elements, respectively designated 46.1, 46.2, 46.3.

Various options exist for the manner in which the connecting elements establish connections between the wales. In one alternative (see FIG. 10), adjacent pairs of wales (e.g., 44.1 and 44.2) are linked by a connecting element (46.1). Using the inlay technique, this connecting element adopts a waveform pattern, traversing through, and being trapped by, the overlap or underlap of adjacent wales.

Each connecting element 46 can be an inelastic yarn or composite yarn. Inclusion of a composite yarn, in the examples, can bestow a variety of radial expansion options to the graft. This is in addition to the radial expansion achieved through the selection of the knit structure.

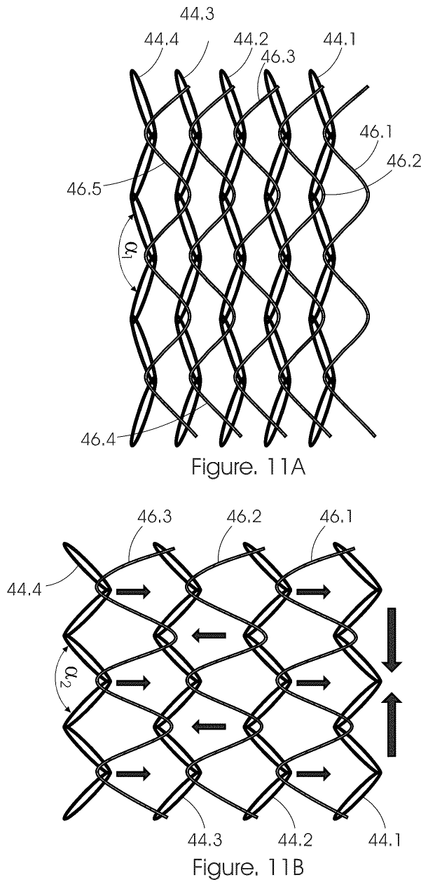

A benefit of pillar stitching with the connecting element inlay is that it results in a scaffold structure with a large allowable radial expansion range before a limiting diameter is reached. This is best illustrated with reference to a relaxed configuration of the knit shown in FIG. 11A against a stretched configuration shown in FIG. 11B and the change in the angle α. A disadvantage is that this large radial expansion range may come with significant longitudinal foreshortening.

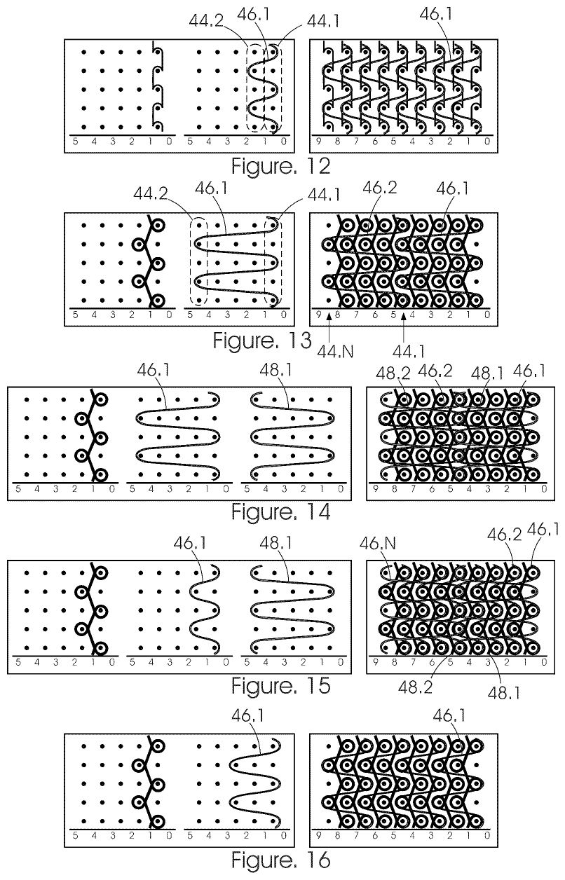

FIG. 12 is a lapping diagram showing the construction of the knitted fabric 40.1 already described and illustrated in FIGS. 10, 11A and 11B.

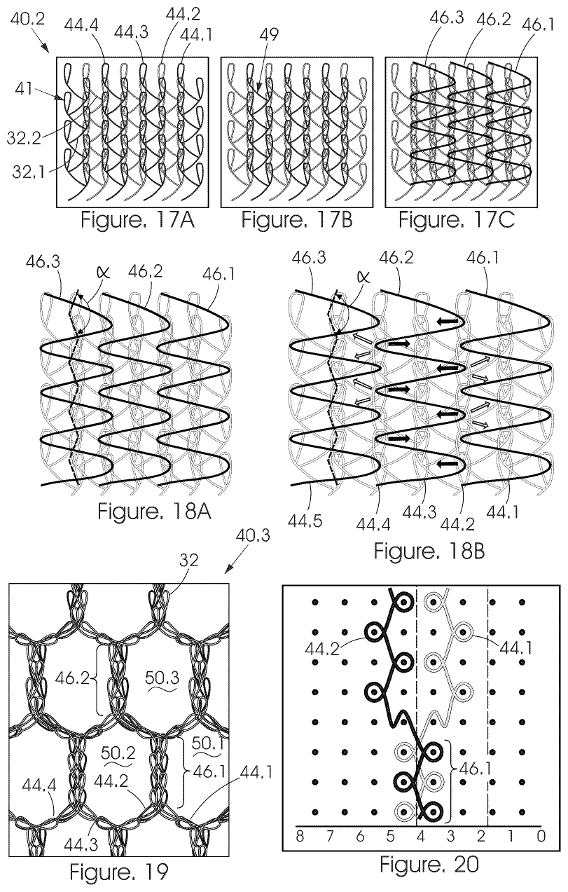

Tricot Stitch: In a second example of a knitted fabric 40.2, illustrated in FIGS. 17A, 17B and 17C, the knit pattern is a tricot stitch knit pattern. Wales (44.1, 44.2, etc.) are established using a single yarn system, where guide bars traverse between adjacent wales, resulting in stitches along each wale. These stitches are alternatively formed by a warp yarn 32.1 from a first set and a warp yarn 32.2 from a second set, thereby creating interconnected wales. In this instance, the connecting elements consist of cross-links 49 generated by each warp yarn as it spans between a pair of wales.

The tricot stitch creates a stable structure without the need for additional connecting elements. However additional connecting elements, in the form of inlay yarns 46 can be introduced for additional stability and a desired limiting diameter.

An advantage of introducing these inlay elements 46.1, 46.2, and 46.3 (see FIGS. 18A and 18B) is that it allows for an overall diameter restriction without significant alterations in the angle (α) between successive courses, in contrast to the substantial reduction in this angle (α) observed in the pillar stitch embodiment. Achieved by the forces exerted by the connecting elements, as the scaffold expands radially toward the limiting diameter, and counteracted by the structure of the connecting base pattern, this manifests in diametric restriction with limited foreshortening, a significant advantage.

FIG. 16 is a lapping diagram showing the construction of the knitted fabric 40.2 already described and illustrated in FIGS. 17A, 17B, 17C, 18A and 18B.

Similar to the pillar stitch embodiment 40.1 illustrated in FIG. 10, each connecting element (46.1, 46.2, 46.3), a composite yarn for example, adopts a sinusoidal pattern as it interlaces with, and connects, the pair of wales. In this example, the connecting elements are positioned radially spaced apart and phase-aligned. They stretch between the pair of wales (44) which are separated by a single interposing wale.

FIG. 13 illustrates another alternative wherein each connecting element (46.1, 46.2, . . . ) is configured to bridge and interlink non-consecutive wales (44.1, 44.2) which are separated by three intervening wales.

FIG. 14 illustrates a third alternative, in which a second plurality of connecting elements (48.1, 48.2, . . . ) is introduced with a third yarn system allowing for the connecting elements of the first (46) and the second (48) plurality to extend in opposing directions in an alternating mirrored configuration.

In a last example, the connecting elements of the first (46.1, 46.2, etc.) and the second (48.1, 48.2, etc.) pluralities also extend in opposite directions, with the peaks and troughs of the elements of the first and second plurality in phase. This example differs in the separation of the respective wale pairs connected by the first and second pluralities. The connecting elements of the first plurality connect adjacent wales, whilst the connecting elements of the second plurality connect wales which are spaced apart by three interposing wales (see FIG. 15).

The diverse connecting element inlay configurations outlined in the context of the tricot knit fabric 40.2 can be applied to the pillar knit fabric 40.1. Although the inlay configurations depicted in FIGS. 13 to 16 are better suited for incorporation to the tricot knit rather than the pillar knit due to a more stable structure resultant structure.

For the sake of completeness, the connecting elements (46, 48) can be added using a weft insertion, as an alternative to the inlay technique, in which the connecting element is added perpendicular to the production direction.

Auxetic net: In a third example, designated 40.3 and illustrated in FIG. 19, the knitting pattern adopts a net-like structure, as depicted in FIG. 19. This net structure is created by forming wales (44.1, 44.2, . . . ) of warp elements 32. In this configuration, wales on opposite sides (such as 44.3 and 44.1 in this example) of a central wale (44.2) interlace with it along alternating interlaced sections (46.1, 46.2). Specifically, wale 44.1 interlinks with wale 44.2 along section 46.1, while wale 44.3 interlaces with wale 44.2 along section 46.2.

In this net-like structure, when the underlaps cross, wales are drawn together, forming net pillars 46 (the interlaced sections described above). Conversely, when the underlaps do not cross, the wales remain separate, creating net openings (50.1, 50.2, 50.3, . . . ) (see FIG. 20).

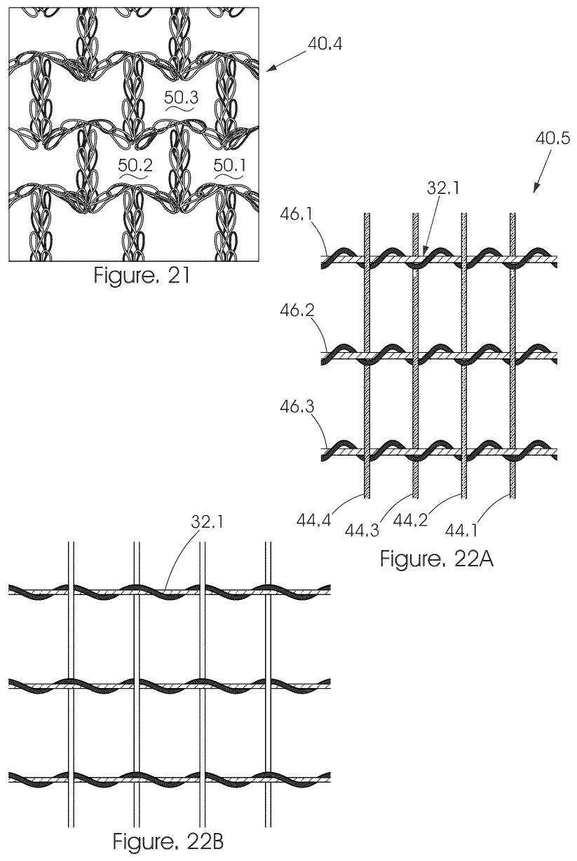

In a variation of this embodiment, designated 40.4 and depicted in FIG. 21, alterations are made to the net opening geometry with the objective of reducing the longitudinal distance between successive net pillars 46. This modification transforms the net opening 50 from a hexagonal shape to a dog bone or bowtie shape. Such a geometric change is implemented during production through either a) manipulating the connecting pattern or b) incorporating an elastic warp element that spans the net opening in the longitudinal or production direction.

Alternatively, achieving the desired net opening geometry can involve postprocessing steps. For instance, the knitted surface may undergo longitudinal compression and subsequent heat setting to fix the modified opening shape. The dog bone shapes of the net openings are configured to confer auxetic properties on the scaffold, wherein radial expansion also encourages longitudinal expansion.

These auxetic net-like embodiments (40.3, 40.4) are beneficial in that they eliminate the foreshortening problem, allowing for longitudinal compliance, and more closely resembles the behavior of native tissue.

### General

The yarns making up the warp elements and the connecting elements can have a linear density of from 20 decitex (dtex) to 80 dtex.

The yarns of the weft or connecting elements may be the same as the warp elements or may have a differing linear density, material, spin, or construction (including composite covered yarns) in order to impart the desired limiting diameter.

In the preceding embodiments, the total number of wales per cm is between 3 and 50. Where a wale is a row of loops in the production (functionally longitudinal) direction, the number of wales per cm is a measure of latitudinal knit density.

The knit structure can be formed with course density between 6 and 50 courses per cm. Where a course is a row of loops perpendicular to the production direction, the number of courses per cm is a measure of longitudinal knit density.

The warp and connecting elements can be configured to allow for unrestricted radial expansion in one range, whilst limiting radial expansion at a second strain.

### Weave

As an alternative to the knitted fabrics (40.1, 40.2, 40.3, 40.4), the fabric can be formed using a basic weave pattern. This weave pattern fabric is designated 40.5 and illustrated in FIGS. 22A and 22B. In one variation, the weft yarns, respectively designated 46.1, 46.2, 46.3, 46.4) are composite yarns 32.1, which are introduced to allow radial compliance before the engagement of the inelastic element.

As mentioned above, the composite yarn 32.1 can be produced with the number of twists of the covering yarn 36 between 25-1500 twists per meter.

The woven fabric 40.5 when configured with the weft elements being a covered yarn will have an allowable degree of radial expansion in which only the elastic core is under strain, and a target radial expansion at which the inelastic cover yarn is engaged, thereby limiting radial compliance beyond a certain radial strain.

In another embodiment (not shown) the warp elements (44.1, 44.2, 44.3, 44.4) of the woven fabric 40.5 are also composed of a composite covered yarn. The construction of the first (weft) covered yarn and the second (warp) covered yarn may be different to confer differing degrees of radial and longitudinal expansion.

The woven fabric can be constructed in a basic weave structure, as illustrated, or a Leno weave structure (a technique where paired warp yarns twist around the weft yarn to create a more open and flexible fabric), with covered yarns in the weft and/or warp direction.

### Compliant Component

While the mechanical component plays a crucial role in constraining the expanded state, the compliant component must allow the tubular body to expand while also facilitating a biased return from the expanded state to the native state. The optimal functioning of the vascular graft 10, with the intended benefit of controlled compliance, relies on the interplay between the compliant and mechanical components.

Similar to the mechanical component, various essential characteristics, whether individually or in combination, configure the compliant component to fulfil its functional purpose of compliance and recoil. These attributes include the layering configuration, as described earlier (referring to the number of layers in the compliant component), the material of manufacture (comprising the material constituting the compliant component), and the method of application and adhesion (how the compliant component is applied to the graft).

Material of Manufacture: The compliant component comprises an elastic biocompatible synthetic polymeric material. As illustrated in FIGS. 2 to 6, the vascular graft exists in various iterations with diverse layering configurations, and the elastic polymeric material composition differs between the first and second layers.

The compliant component is made from thermoplastic polyurethane (TPU) or a thermoplastic polyurethane silicone co-polymer (TPU-Silicone). This material is selected primarily for its elasticity, impermeability, biostability, and mechanical and tensile strength (sufficient to provide erosion resistance and to retain embedded stents).

All layers (26 and 28 or 28.1 and 28.2) within the compliant component are functionally compliant, regardless of their position in the layering structure. However, only a specific layer, the tie-layer, requires no recoil functionality, needing only to possesses low tensile strengths so as to not impede on graft compliance and adhesive properties to adhere to the mechanical component.

The TPU or TPU-silicone is produced:
through the synthesis of polycarbonate diols and aliphatic or aromatic di-isocyanates, referred to here as “a polycarbonate TPU”, or
from thermoplastic silicone urethane copolymers, which involve crosslinked silicone and TPU, wherein these copolymers are synthesized using diisocyanates and hydroxyl-terminated siloxanes from the diisocyanate family, or
with polyester or polyether polyols or aliphatic or aromatic di-isocyanates.

The polycarbonate TPUs and the TPU-Silicones are highly compliant and elastic.

Polycarbonate TPUs or TPU-Silicones, either aliphatic or aromatic, demonstrate a Shore hardness of 65A to 95A. They possess an ultimate tensile strength falling between 27 MPa and 60 MPa, an ultimate elongation ranging from 300% to 1200%, and a tensile strength of 2 MPa to 7 MPa at a 100% elongation rate. Additionally, these TPUs have a flexural modulus within the range of 6 MPa to 22 MPa.

In order to enhance biostability and increase elasticity, TPU-Silicones can be adapted to include silicone, in a concentration of 5%-50% (wt/wt), along the polymer backbone or as covalently bonded end groups.

Polyester or polyether polyols or aliphatic or aromatic di-isocyanates TPUs are compliant, have good adhesive/cohesive properties, but are relatively soft, having a Shore hardness ranging from 42A to 70A, an ultimate tensile strength within the range of 6 MPa to 20 MPa, a tensile strength falling between 1 MPa and 3 MPa at a 100% elongation rate and an ultimate elongation varying from 500% to 900%.

A layer of the compliant component, for example the first layer 26, can include longitudinal zones (52.1, 52.2, 52.3) of varying durometer along the length of the graft body 12 (see FIG. 26), brought about by using different materials in each longitudinal zone. This configuration establishes specific longitudinal compliance zones to accommodate varying radial compliance needs along the length of the device. For instance, the proximal and distal zones (52.1, 52.3) exhibit greater stability, increased hardness, and enhanced suturability, with a durometer ranging between 85-95A. Meanwhile, the durometer of an intermediate zone falls within the range of 65-85A.

Method of Application: The elastic polymeric material of the compliant component can be applied to the underlayer, for example the mechanical knitted layer 22, to cover, bind to, or encapsulate (where solid) this layer.

The compliant component can be a solid preform made by electrospinning (providing a filamentous tubular structure), or by extrusion, dip molding or spray molding (providing a uniform sheet-like or laminar tubular structure). The solid preform is then applied or bonded to the underlayer.

Alternatively, the compliant component can be electrospun directly onto the underlayer. Further alternatively, the compliant component can be dip or spray molded directly onto the underlayer to permeate the underlayer, if the underlayer is the mechanical component, to encapsulate the mechanical component.

The choice of application method largely depends upon the construction of the mechanical component-dip molding is not preferred if the mechanical component has elastic yarns- and the type of polymeric material making up the compliant component-if the polymeric material is not solution grade, extrusion (melting) is preferred.

When applied to the underlayer as a solid preform, the elastic polymeric material of the compliant component has a thicknesses from 30 μm to 400 μm.

With a chosen Polycarbonate TPU or TPU-Silicone, with the thickness range mentioned above, the solid preform will the necessary properties for elastic recoil, being:
an ultimate elongation of 500% to 1200%,
a tensile strength of 2 MPa to 20 MPa, and
an elastic modulus of 0.2 MPa to 3 MPa in strains up to 200%.

The elastic polymeric material of the compliant component can be electrospun onto the underlayer (in this example, the mechanical component 22) in a first layer (28.1) and a second layer (28.2). The first layer can have a thicknesses from 30 μm to 400 μm, and the second layer can have a thickness of 10 μm-400 μm.

The fiber diameters of the electrospun filaments will be from 0.9 μm to 4 μm.

In the examples, illustrated in FIGS. 27, 28A and 28B, the electrospun fibers of the first layer 28.1 are laid down in a random meshed configuration, whilst the fibers of the second layer 28.2 are laid down in a unidirectional manner. Placing the fibers unidirectionally in the circumferential direction of the tubular body 12 (see FIGS. 27 and 28A) will complement the mechanical component, restricting expansion. Conversely, orienting the fibers longitudinally will constrain longitudinal compliance while improving radial elasticity. Positioning the fibers at an angle (see FIG. 28B) will yield properties intermediate to these extremes, with the specific characteristics influenced by the chosen slant angle. In this way, the second layer will help prevent over expansion in the compliant component, a function that is absent in the sheet like/laminar tubular compliant components.

### External Brace

To limit the extent to radial compliance, the graft 10 of any embodiment described above can include a brace 50. In the example of FIG. 29A, the brace is a helical filament 50.1 which is helically wound about, or wound about and engaged to, an outer surface of the tubular body 12. The helical filament can be a composite or inelastic yarn (32).

In another example, the brace is a frame made from the same elastic polymeric material constituting the compliant component. But instead of being applied as a sheet or layer, it is printed by inkjet directly to an underlayer (either 22 or 28) or preprinted and then applied. Inkjet printing allows for intricate patterns to be created for the frame, as exemplified in FIGS. 29B, 29C, and 29D, and respectively designated 50.2, 50.3 and 50.4. As an alternative, in FIGS. 29D and 29E, the frame 50.4 and 50.5, being in this example, a series of struts or waveform rings, can be made of super elastic metal alloy such as, for example, nitinol.

In making the brace from an elastic polymeric material, these latter examples incorporate a mechanical element that is part of the compliant component. The advantage here is in introducing a lock-out property, which is associated with the mechanical component, through the compliant material, albeit with a bias.

1.-46. (canceled)

47. A composite compliant vascular graft material for replacement of a portion of a vessel, comprising:
a tubular body extending between a proximal end and a distal end, the tubular body having a lumen and comprising a composite compliant material adapted to permit expansion of the tubular body from a native state to an expanded state and then to return to the native state in response to pressure changes in the lumen, the composite compliant material comprising:
a mechanical component adapted to limit the expanded state; and
a compliant component adapted to promote the return of the tubular body to the native state, wherein:
the composite compliant material has anisotropic mechanical properties configured to facilitate radial and longitudinal expansion of the tubular body to the expanded state and radial and longitudinal expansion recoil to the native state in response to the pressure changes.

48. The composite compliant vascular graft material according to claim 47, wherein the mechanical component is adapted to limit at least one of:
an increase in a radial dimension of the expanded state, or
a decrease in a longitudinal dimension of the expanded state.

49. The composite compliant vascular graft material according to claim 48, wherein the mechanical component is adapted to accommodate an increase in the longitudinal dimension of the expanded state.

50. The composite compliant vascular graft material according to claim 48, wherein the mechanical component comprises a framework of filamentous elements.

51. The composite compliant vascular graft material according to claim 50, wherein the filamentous elements comprise yarns, including elastic yarns, inelastic yarns, or a combination of elastic and inelastic yarns.

52. The composite compliant vascular graft material according to claim 50, wherein the framework is constructed of yarns intertwined in a knit structure or a weave structure.

53. The composite compliant vascular graft material according to claim 52, wherein the framework is constructed of yarns intertwined in the weave structure, and wherein the weave structure comprises weft yarns and warp yarns, adapted to adjust the extent of radial expansion or longitudinal dimension of the expanded state.

54. The composite compliant vascular graft material according to claim 52, wherein the framework is constructed of yarns intertwined in the knit structure, and wherein the knit structure comprises a warp knit or a weft knit.

55. The composite compliant vascular graft material according to claim 47, wherein the compliant component comprises at least a first layer of the tubular body that is engaged to the mechanical component or that encapsulates the mechanical component.

56. The composite compliant vascular graft material according to claim 55, wherein the compliant component comprises a tie layer of the tubular body that is engaged to the first layer or the mechanical component.

57. The composite compliant vascular graft material according to claim 56, wherein the compliant component comprises at least a second layer of the tubular body that is engaged to an underlayer being the first layer, the tie layer, or the mechanical component.

58. The composite compliant vascular graft material according to claim 57, wherein the compliant component is engaged as the first layer, the second layer, or the tie layer to the underlayer, or the compliant component is applied directly as the first layer, the second layer, or the tie layer to the underlayer to cover or to encapsulate the underlayer.

59. The composite compliant vascular graft material according to claim 58, wherein the compliant component is a laminar layer or a filamentous layer.

60. The composite compliant vascular graft material according to claim 59, wherein the compliant component is the filamentous layer, and wherein the filamentous layer comprises at least one of:
a third layer comprising filaments orientated in multiple directions, and
a fourth layer comprising filaments orientated unidirectionally in a circumferential direction or an axial direction of the tubular body.

61. The composite compliant vascular graft material according to claim 60, wherein the filaments of at least one of the third layer or the fourth layer are electrospun.
